(19) United States
(12) Patent Application Publication
(10) Pub. No.: US 2021/0381024 A1
(43) Pub. Date: Dec. 9, 2021

(54) Personalized System for Restoring the Gastrointestinal Tract Microbiome

(71) Applicant: Locus IP Company, LLC, Solon, OH (US)

(72) Inventors: Sean FARMER, Ft. Lauderdale, FL (US); Ken ALIBEK, Solon, OH (US); Albina TSKHAY, Solon, OH (US)

(21) Appl. No.: 17/284,985

(22) PCT Filed: Oct. 20, 2019
(86) PCT No.: PCT/US2019/057106

§ 371 Date: Apr. 13, 2021

Related U.S. Application Data
(60) Provisional application No. 62/751,098, filed on Oct. 26, 2018.

(51) Int. Cl. C12Q 1/04; A61K 35/741; A61K 35/744; A61K 35/745; A61K 35/747; A61K 35/742; C12N 1/20; A61K 38/12

(52) U.S. Cl. CPC C12Q 1/04; A61K 35/741; A61K 35/744; A61K 35/745; A61K 2035/115; A61K 35/742; C12N 1/20; A61K 38/12; A61K 35/747

(57) ABSTRACT

The present invention provides methods for enhancing the health of a subject using microorganisms indigenous to the subject's own gut microbiome. In one embodiment, the microbial population of a subjects GI tract and/or appendix is sampled and sequenced to identify and determine the population percentage of species. The beneficial species are used to inoculate novel, distributed fermentation systems. The beneficial species are cultivated to a high concentration and reintroduced into the subjects GI tract in order to restore the gut microbiome to a balanced and healthy state.

### CROSS-REFERENCE TO RELATED APPLICATIONS

This application claims priority to U.S. Provisional Patent Application No. 62/751,098, filed Oct. 26, 2018, which is incorporated herein by reference in its entirety.

### BACKGROUND OF THE INVENTION

The gut microbiota is an essential ecosystem within the human body that participates in a large number of vital functions. This system contains a variety of taxa, including bacteria, eukaryotes, viruses, and archaea. Trillions of these organisms build a complex symbiotic relationship, providing a number of benefits to the host, including aiding in normal physiological functions and disease resistance (Clemente 2012).

The composition of each person's microbiota is unique. Twins have been shown to share less than 50% similarity of bacterial taxa, and even less viral similarity (Turnbaugh 2010). Many of the species present in the gut microbiome are bacteria belonging predominately to the phylotypes Bacteroidetes and Firmicutes, while some others, including Actinobacteria, Proteobacteria and Verrucomicrobia, are minor constituents. Methanogenic archaea (mainly Methanobrevibacter smithii), eukaryotes (mainly yeasts) and viruses (mainly phages) can also be present. While these groups tend to be universally present within the gut microbiome, their relative proportions and the particular species vary between individuals (Luzopone 2012). This difference may be determined by genetic influences (Benson 2010).

An individual's health is closely linked to the health of the gut microbiome. In other words, changes in the gut microbiome are often either a cause or an effect of changes in an individual's health. For example, intestinal microflora play an important role in metabolic functions, as well as digestive, immune, endocrine and cardiovascular system functions. When the gut microbiome is disrupted or unbalanced, for example, due to illness, dysbiosis, the presence and/or overgrowth of pathogenic and/or commensal organisms, the presence of a parasite, the use of antibiotics, a food allergy or sensitivity, the implementation of a colonoscopy, or some other influence, these and other body system functions can also be disrupted.

Intestinal bacteria are the most important components of mammalian metabolic and digestive processes. These microbes aide in enterohepatic circulation, and help with the digestion and assimilation of energy sources and essential trace elements into the GI system. Additionally, anaerobic bacteria of the gut break down and/or ferment heavy carbohydrate compounds, such as dietary fiber, into short-chain fatty acids, such as acetate, propionate and butyrate. Gut microbes also aide in the regulation of fat storage, blood sugar levels, and metabolism of several vitamins. For example, the intestinal microflora synthesizes vitamin K, which is a necessary cofactor in the production of prothrombin and other coagulation factors. Additionally intestinal bacteria synthesize biotin, vitamin B12, folic acid and thiamine.

There is also a relationship between the gut microbiota and the immune system. The human intestine contains more immune cells than all other parts of the body. A balanced gut microbiome regulates many functions of the immune system, including the activation and regulation of immune cells, proliferation of regulatory T-cells, neutrophil activation, and migration of the monocytes and macrophages. Thus, disturbances in the gastrointestinal tract may lead to immune disorders, infections and mis-regulation of pro- and anti-inflammatory processes. Furthermore, various other health concerns, ranging from autoimmune diseases to clinical depression and obesity, can be related to immune dysfunction, which can be linked to an unbalanced gut microbiome.

A balanced gut microbiome is also important for hormone regulation. For example, serotonin, which is a hormone that participates in processes such as sleep, mood, sexual affection, production of breast milk, respiration, communication, production and regulation of melatonin and adrenaline, and many others, is largely (i.e., more than 90%) produced in the GI tract by enterochromaffin cells. The microbial makeup within the GI tract may play a role in proper serotonin production and regulation.

Additionally, an imbalance of the GI microbiome can affect the cardiovascular system. Disruptions in the gut microbiome contribute to an increase in the appearance of plaques and the progression of atherosclerosis. Furthermore, the GI tract affects the regulation of insulin, the resistance to which can lead to diabetes—a condition that greatly increases the risk of cardiovascular disease. Even further, disrupted gut flora can lead to accumulation of adipose tissue in organs such as the liver and heart, which also increases the risk of diseases associated with these organs.

In addition to the digestive, metabolic, endocrine and cardiovascular systems, changes in the gut microbiome are also associated with a number of autoimmune diseases, and may be a cause and/or an effect of these pathologies. For example, altered microbiota are thought to play a role in initiation and progression of inflammatory bowel syndrome (IBS), Crohn's disease, and ulcerative colitis through triggering of autoimmune responses and inflammation. The autoimmune disease, celiac disease, may also be triggered by bacterial and/or viral infections that augment gut mucosal responses to gluten. Furthermore, Type 1 diabetes is also caused by immune system alterations, which may be caused and/or progressed by gut microbiome alterations that affect the mucosal lining of the intestines and gut permeability.

The intestinal microbiome may also be key to the etiology of colorectal cancer in at least two ways: through the pro-carcinogenic activity of microbial pathogens and through the influence of the metabolome. For instance, suppression of inflammation and cancerous cells may be achieved by the presence of short-chain fatty acids, acetate, propionate and butyrate produced by beneficial gut microbes, while other compounds, such as secondary bile acids, may induce carcinogenesis. Pathogenic microbes may also cause DNA alterations that could lead to oncologic diseases.

There is also evidence for an association between altered gut microbiota and neurodevelopmental (e.g., autism) and neurodegenerative (e.g., Alzheimer's) diseases. For example, Alzheimer's disease is characterized by accumulation of amyloid-β fibrils in the brain. Gut microbiota were shown to produce a significant amount of amyloids and lipopolysaccharides, which affect the neural pathways and production of cytokines, thus contributing to the disease.

Additionally, anxiety and sensory sensitivity experienced by patients with autism have been found to correlate with GI problems, and in fact, about 70% of children with autism have GI problems. Furthermore, children with autism show a higher level of Clostridium histolyticum than healthy children, and certain microbes, such as Desulfovibrio bacteria may play a role in the development of regressive autism.

The microbial community within the gut can also fluctuate depending on what stage of life a host is in. It has been found that gut microbiota profiles of elderly people are different from those of healthy adults. This could be attributed to changed lifestyle and dietary schedule, lesser mobility, weakened immune strength, reduced intestinal and overall functionality, altered gut morphology and physiology, recurrent infections, hospitalizations, and use of medications, all of which are associated with senescence. Though it is unclear whether microbiota changes are a cause or an effect of aging, the incidences of comorbidities associated with gut microbiota tend to increase as the host grows older (Nagpal 2018).

Currently, the most common way to repair microbiota is usage of probiotics, such as L. acidophilus, L. casei, L. rhamnous, L. bulgaricus, B. bifidum, S. thermophilus, and others. Usually, probiotics contain one or several live strains of bacteria that are capable of temporarily populating the gut and providing relief to symptoms such as diarrhea, constipation, bloating or nausea. Nonetheless, the effects of probiotics typically do not last for extended periods of time.

An additional method of repairing the gut microbiome is through fecal bacteriotherapy, or fecal transplantation. This method involves introducing saline-diluted fecal matter from a healthy donor into a patient's gastrointestinal tract via a nasoduodenal cathether or enema. In many cases, the donor is a relative of the patient. Thus far, fecal transplant has primarily been used to treat Clostridium difficile enterocolitis; however, this method has not been as effective for treating other diseases, such as IBS and Crohn's disease. Additionally, the method is limited by the ability to find a healthy donor, the safety of the method, side effects including abdominal discomfort, bloating, flatulence, diarrhea, constipation, borborygmia, vomiting, transient fever, bleeding and bacteremia, and risk of transmission of infection and/or some chronic diseases.

The composition of the gut microbiome of every person is individual, and is possibly predisposed by genetic factors. When the microbiome of an individual is disrupted or unbalanced, or when an individual is afflicted with a disease or condition that causes such disruption or imbalance, the individual would benefit from improved methods of restoring the gut microbiome back to a balanced state.

### BRIEF SUMMARY OF THE INVENTION

The present invention provides improved methods for enhancing the health of a subject through restoration of the subject's gut microbiome. The present invention also provides systems and methods for cultivating microorganisms for use in restoring a subject's gut microbiome.

In one embodiment, the present invention provides personalized methods of restoring a subject's gut microbiome. More specifically, the methods comprise re-inoculating the subject's gastrointestinal (GI) tract with beneficial microorganisms isolated from the subject's own unique GI microbiome. Advantageously, the methods provide for personalized treatments for a variety of health conditions that are associated with the health and balance of the intestinal microbiome.

The methods of the present invention utilize indigenous microorganisms present in a subject's GI tract. In certain embodiments, the subject's gut microbiome has been disrupted or unbalanced. The disruption or imbalance can be a result of, for example, illnesses, infection, aging, dietary factors (e.g., food sensitivities, changes in eating and/or nutritional habits), immune system changes, treatment with antibiotics, or procedures, such as appendectomies and/or colonoscopies.

In one embodiment, the method comprises taking a sample from the subject's GI tract, wherein the sample comprises a microbial community. In one embodiment, the microbial community is a representation of the entire microbial community within the subject's GI tract.

In one embodiment, the sample is taken from the subject's appendix.

The sample is then analyzed to determine the identity of microbial species present within the microbial community, and to determine the ratio of each species with respect to the other species of the microbial community. Analysis can comprise standard methods in the art, such as, for example, DNA sequencing, DNA fingerprinting, ELISA, and cell plating.

After analyzing the sample, undesirable microbial species are determined, including overgrown commensal bacteria, certain yeasts and fungi, as well as pathogens and parasites. Additionally, beneficial species are determined and isolated.

The isolated beneficial species is/are then cultivated, either separately or together (e.g., symbiotically) in order to produce a microbial culture with an increased cell count and/or cell concentration. In certain embodiments, the cell concentration is increased to about 1×10⁶ to about 1×10¹³ cells/gram. In preferred embodiments, cultivation is carried out using a small scale, modified solid state fermentation system.

The increased concentration microbial culture is then reintroduced back into the GI tract of the subject. In preferred embodiments, the subject's GI tract is cleansed using, for example, colon hydrotherapy, prior to reintroduction of the culture. The hydrotherapy can be performed once or multiple times, as determined by a skilled healthcare provider.

As the beneficial microorganisms grow, their increased concentration allows them to outcompete and/or control the undesirable microorganisms, thus restoring the gut microbiome to a healthy, balanced state. In certain embodiments, the method further comprises administering prebiotics to the subject before, concurrently with, and/or after reintroduction of beneficial microbes to provide an enabling environment for the beneficial microorganisms to grow, and to decrease the amount of time required to restore the gut microbiome.

Restoring a subject's gut microbiome can comprise balancing an unbalanced gut microbiome, regardless of whether the imbalance is a cause or an effect of a disease or another change to the subject's health status. Restoration preferably comprises decreasing the number of overgrown commensal microorganisms and/or pathogenic microorganisms in the GI tract, and/or increasing the number of beneficial microorganisms in the GI tract. The composition of the gut microbiome (e.g., the species and proportions of different microorganisms within the GI tract) is unique to every individual, and is possibly predisposed by genetic factors; thus, whether or not a microorganism is commensal, harmful or beneficial, and what proportion of the microbiome each species comprises, is unique to an individual.

In one embodiment, the method further comprises introducing a microbial growth by-product that can further enhance the restorative capabilities of the present methods. The growth by-products can include those that are produced by the beneficial microbes of the reintroduced microbial culture, or they can be applied in addition to those produced by the beneficial microorganisms.

In one embodiment, the growth by-products are biosurfactants, enzymes, biopolymers, solvents, acids, proteins, amino acids, or other metabolites that can be useful for, for example, controlling undesirable microorganisms or encouraging the growth of beneficial microorganisms. In a specific embodiment, the growth by-product is a biosurfactant selected from glycolipids (e.g., sophorolipids, rhamnolipids, trehalose lipids, cellobiose lipids and mannosylerythritol lipids) and lipopeptides (e.g., surfactin, iturin, fengycin, arthrofactin and lichenysin).

In certain embodiments, the present invention can be used to enhance a subject's overall health and well-being. In one embodiment, the present invention can be used to reduce the severity of health conditions that result from aging and/or senescence, wherein the subject is a middle-aged or elderly person, e.g., 50 years of age or older.

In one embodiment, the present invention can be used to enhance the functioning of a body system, tissue or organ, such as metabolic functions, the digestive system, the immune system, the endocrine system, and the cardiovascular system.

In one embodiment, the present invention can be used to treat a health condition that is a cause and/or a result of a disrupted or unbalanced gut microbiome, such as, for example, Irritable Bowel Syndrome, Type 1 diabetes, other autoimmune disorders, colorectal cancer, and neurodevelopmental and neurodegenerative diseases.

In preferred embodiments, the present invention provides methods of cultivating a microorganism and/or a microbial growth by-product using a modified version of solid state fermentation, or “matrix fermentation.” Advantageously, the cultivation methods can be scaled up or down in size. In preferred embodiments, the systems, methods and materials are useful for cultivating solid-state microbe-based products at cell concentrations of 1×10⁶ up to 1×10¹³ cells per gram.

In preferred embodiments, the method of cultivating a microorganism and/or producing a microbial growth by-product comprises: spreading a layer of a solid substrate mixed with water and, optionally, nutrients to enhance microbial growth, onto a tray to form a matrix; applying an inoculant of a microorganism onto the surface of the matrix; placing the inoculated tray into a fermentation reactor; passing air through the reactor to stabilize the temperature between 25-40° C.; and allowing the microorganism to propagate throughout the matrix.

The inoculant preferably comprises a microorganism that has been isolated from a subject's GI tract and has been determined to be beneficial.

The inoculated trays can then be placed inside a fermentation reactor and incubated for an amount of time that allows for the microorganism to reach a desired concentration, or to reach from 50-100% sporulation, preferably from 1 day to 14 days, more preferably, from 2 days to 10 days. In some embodiments, the microorganisms will consume either a portion of, or the entirety of, the matrix substrate throughout fermentation.

The culture and remaining substrate can be harvested from the trays, then blended together to produce a microbial slurry. In one embodiment, the microbial slurry is milled, micronized and/or dried to produce a dry microbial culture that contains microorganisms and substrate. The microbial slurry can also be dissolved in water in a mixing tank to form a microbial culture in liquid form.

Activation and/or germination of microbial spores can be enhanced, either during cultivation or at the time of application, by adding L-alanine in low (micromolar) concentrations, manganese or any other known germination enhancer.

In certain embodiments, the present invention provides high concentration microbe-based products for use in restoring a subject's GI microbiome, wherein the microbe-based product is a microbial culture having a cell concentration of about 1×10⁶ to 1×10¹³ cells per gram, preferably 1×10⁸ to 1×10¹³. Organisms that can be cultured according to the present invention can include, for example, yeasts, fungi, bacteria, and archaea that have been sampled and identified from the subject's GI tract and/or appendix. In preferred embodiments, the microorganisms are bacteria.

The high concentration microbe-based products produced according to the fermentation methods of the present invention can comprise the substrate, microorganisms and/or microbial growth by-products, as well as nutrients for microbial growth. The microorganisms can be viable or in an inactive form. They can be in the form of vegetative cells, spores, conidia, mycelia and/or a combination thereof.

In one embodiment, the growth by-products of the microorganisms are biosurfactants, enzymes, biopolymers, solvents, acids, proteins, amino acids, or other metabolites that can be useful for, for example, gut health, control of undesirable microorganisms and growth of beneficial microorganisms. In a specific embodiment, the growth by-product is a biosurfactant selected from glycolipids (e.g., sophorolipids, rhamnolipids, trehalose lipids and mannosylerythritol lipids) and lipopeptides (e.g., surfactin, iturin, fengycin and lichenysin).

Advantageously, the methods of the present invention reduce the risks that are associated with fecal transplants, including, for example, rejection of transplanted microbiota and transmission of infection, through the use of indigenous beneficial microorganisms in the subject's GI tract. Additionally, the microbial population of an individual can vary greatly from that of another individual based upon, for example, their genetics; thus, the use of indigenous microorganisms helps to reduce the time needed, for example, through trial and error, to create an efficient microbial population because microorganisms that are suited for a particular individual's GI tract are already present. Furthermore, use of the subject's indigenous beneficial microflora increases the chances of sustained, long-term gut health, as compared to, for example, short-lived probiotics.

### DETAILED DESCRIPTION

The present invention provides methods for enhancing the health of a subject through restoration of the subject's gut microbiome. The present invention also provides systems and methods for cultivating microorganisms for use in restoring a subject's gut microbiome.

In one embodiment, the present invention provides personalized methods of restoring a subject's gut microbiome. More specifically, the methods comprise re-inoculating the subject's gastrointestinal (GI) tract with beneficial microorganisms isolated from the subject's own unique GI microbiome. Advantageously, the methods provide for personalized treatments for a variety of health conditions that are associated with the health and balance of the intestinal microbiome.

Selected Definitions

The subject invention involves production and use of “microbe-based compositions,” which comprise components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites (e.g., biosurfactants), cell membrane components, proteins, and/or other cellular components. The microbes may be intact or lysed. The cells may be absent, or present at, for example, a concentration of 1×10⁴, 1×10⁵, 1×10⁶, 1×10⁷, 1×10⁸, 1×10⁹, 1×10¹⁰, 1×10¹¹, 1×10¹², 1×10¹³ or more CFU/g or ml of the composition.

The present invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied.

The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, and surfactants.

As used herein, the term “plurality” refers to any number or amount greater than one.

As used herein, the term “reduces” means a negative alteration, and the term “increases” means a positive alteration, wherein the negative or positive alteration is an alteration of at least 1%, 5%, 10%, 15%, 20%, 25%, 30%, 35%, 40%, 45%, 50%, 55%, 60%, 70% 75%, 80%, 85%, 90%, 95%, 99% or 100%.

As used herein, the term “reference” means a standard or control condition.

As used herein, the term “salt-tolerant” in reference to a particular microbial strain, means the strain is capable of growing in a sodium chloride concentration of fifteen (15) percent or greater. In a specific embodiment, “salt-tolerant” refers to the ability to grow in 150 g/L or more of NaCl.

As used herein, the term “subject” refers to an animal whose gut microbiome has been disrupted or unbalanced. The animal may be selected from, for example, pigs, horses, goats, cats, mice, rats, dogs, primates (e.g., apes, chimpanzees and orangutans), guinea pigs, hamsters, cows, sheep, birds (e.g., chickens), reptiles, fish, as well as any other vertebrate or invertebrate. The preferred subject in the context of this invention is a human of any sex or gender. The subject can be of any age or stage of development, including infant, toddler, adolescent, teenager, adult, middle-aged and senior. In some embodiments, the subject is a middle-aged or elderly adult, e.g., 50 years of age or older.

As used herein, the term “surfactant” means a surface active compound that lowers the surface tension (or interfacial tension) between two phases. Surfactants act as, e.g., detergents, wetting agents, emulsifiers, foaming agents, and dispersants. A “biosurfactant” is a surface-active substance produced by a living cell.

As used herein, the term “treatment” refers to eradicating, reducing, ameliorating, or reversing a sign or symptom of a health condition, disease or disorder to any extent, and includes, but does not require, a complete cure of the condition, disease or disorder. Treating can be curing, improving, or partially ameliorating a disorder. “Treatment” can also include improving or enhancing a condition or characteristic, for example, bringing the function of a particular system in the body to a heightened state of health or homeostasis.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

Methods of Restoring the Gut Microbiome

In one embodiment, the present invention provides improved methods for enhancing the health of a subject through restoration of the subject's gut microbiome. The present invention also provides systems and methods for cultivating microorganisms for use in restoring a subject's gut microbiome.

As used herein, reference to the “microbiome,” “microbiota,” “microbial community,” “microflora” or “flora” of the “gut” or of the “GI tract” means the population of microorganisms living within a subject's intestines and/or GI tract. In some embodiments, these microorganisms can also be present in the appendix. A healthy, or balanced, gut microbiome is one that comprises a variety of microbial species, with a majority of those species preferably being beneficial to the health of the subject.

As used herein, a “beneficial” microbe is one that is considered mutualistic, or conferring a benefit to its host, rather than one that is merely commensal (existing within the gut in a non-harmful and non-mutualistic coexistence) or one that is harmful and/or parasitic to the host. Benefits can include, for example, digestion of dietary fiber into short-chain fatty acids and synthesis of certain vitamins.

As used herein, a “disrupted” or “unbalanced” gut microbiome is in dysbiosis, where the species of microbes in a subject's gut comprise an amount, percentage or number of non-beneficial microorganisms such that the amount, percentage or number of these non-beneficial microbes results in disease, discomfort, malnutrition, impaired nutrient absorption and other deleterious health consequences. In certain embodiments, the ratio of non-beneficial microorganisms to beneficial microorganisms is 50% or greater.

Non-beneficial microorganisms include, for example, harmful microorganisms, such as pathogens and parasites, as well as commensal organisms that do not directly harm the host, but when overgrown, outcompete beneficial gut microorganisms.

As used herein, “restoring” a disrupted or unbalanced gut microbiome refers to establishing or reestablishing the predominance of beneficial microorganisms within the gut microbial community, or causing the gut microbiome to become healthy and/or balanced.

In one embodiment, the present invention provides personalized methods of restoring a subject's gut microbiome. More specifically, the methods comprise re-inoculating the subject's gastrointestinal (GI) tract with beneficial microbes isolated from the subject's own unique gut microbiome. Advantageously, the methods provide for personalized treatments for a variety of health conditions that are associated with the health and balance of the GI microbiome.

The methods utilize indigenous microorganisms that are present in a subject's GI tract. In certain embodiments, the subject is an animal, preferably a human, whose gut microbiome has been disrupted or unbalanced.

In one embodiment, the method comprises taking a sample from the subject's GI tract, wherein the sample comprises a microbial community. In one embodiment, the sample is taken from the subject's appendix. In one embodiment, the sample comprises a representation of the entire microbial community within the subject's GI tract.

The sample can be collected by means known in the medical arts. For example, the sample can be a stool sample, intestinal mucosal lavage samples, and/or an intestinal and/or appendix tissue specimen collected via endoscopy and/or biopsy.

The sample is then analyzed to identify microbial species present within the GI microbial community, and to determine the ratio of each species with respect to each of the other species of the microbial community. Analysis can comprise standard methods in the art, such as, for example, DNA sequencing, DNA fingerprinting, ELISA, and cell plating.

After analyzing the sample, the identity of undesirable microbial species are determined, including overgrown and/or commensal bacteria, certain yeasts and fungi, as well as pathogens and parasites. Additionally, the identity of beneficial species are determined and isolated.

The isolated beneficial species is/are then cultivated, either separately or together (e.g., symbiotically) in order to produce a microbial culture having an increased cell count and/or cell concentration. In preferred embodiments, cultivation is carried out using a small scale, modified solid-state fermentation system.

The increased concentration microbial culture is then reintroduced back into the GI tract of the subject. In preferred embodiments, the subject's GI tract is cleansed using, for example, colon hydrotherapy, prior to reintroduction of the culture. The hydrotherapy can be performed once or multiple times, as determined by a skilled healthcare provider.

As the beneficial microorganisms grow within the subject's GI tract, their high concentrations outcompete and/or control the undesirable microorganisms, thus restoring the gut microbiome to a healthy, balanced state. In certain embodiments, the method further comprises administering prebiotics to the subject before, concurrently with, and/or after reintroduction of beneficial microbes to provide an enabling environment for the beneficial microorganisms to grow, and to decrease the amount of time required to restore the gut microbiome.

Prebiotics can include, for example, fermentable fibers derived from fructans and xylans, inulin, fructooligosaccharides, xylooligosaccahrides and galactooligosaccharides. Foods known to contain prebiotics include, for example, chicory root, onions, garlic, leek, oatmeal, wheat bran, asparagus, dandelion greens, Jerusalem artichoke, and banana.

Restoring a subject's gut microbiome can comprise balancing an unbalanced gut microbiome, regardless of whether the imbalance is a cause or an effect of a disease or another change to the subject's health status. Restoration preferably comprises decreasing the number of commensal and/or pathogenic microorganisms in the GI tract, and/or increasing the number of beneficial microorganisms in the GI tract. The composition of the gut microbiome (e.g., the species and proportions of different microorganisms within the GI tract) is unique to every individual, and is possibly predisposed by genetic factors; thus, whether or not a microorganism is commensal, harmful or beneficial, and what proportion of the microbiome each species comprises, is unique to an individual.

In one embodiment, the method further comprises introducing a microbial growth by-product that can further enhance the restorative capabilities of the methods. The growth by-products can include those that are produced by the microbes of the beneficial microbial culture, or they can be applied in addition to those produced by the beneficial microorganisms.

In one embodiment, the growth by-products are biosurfactants, enzymes, biopolymers, solvents, acids, proteins, amino acids, or other metabolites that can be useful for, for example, controlling undesirable microorganisms. In a specific embodiment, the growth by-product is a biosurfactant selected from glycolipids (e.g., sophorolipids, rhamnolipids, trehalose lipids, cellobiose lipids and mannosylerythritol lipids) and lipopeptides (e.g., surfactin, iturin, fengycin, arthrofactin and lichenysin).

Advantageously, the methods of the present invention reduce the risks that are associated with fecal transplants, including, for example, rejection of transplanted microbiota and transmission of infection, through the use of indigenous beneficial microorganisms in the subject's GI tract.

Additionally, the microbial population of an individual can vary greatly from that of another individual based upon, for example, their genetics; thus, the use of indigenous microorganisms helps to reduce the time needed through, for example, trial and error, to create an efficient microbial population because microorganisms that are suited for a particular individual's GI tract are already present. Furthermore, use of the subject's indigenous beneficial microflora increases the chances of sustained, long-term gut health, as compared to, for example, short-lived probiotics.

In certain embodiments, the present invention can be used to enhance a subject's overall health and well-being. In one embodiment, the present invention can be used to reduce the severity of senescence- or aging-related conditions, wherein the subject is a middle-aged or elderly person, e.g., 50 years of age or older. Aging-related conditions can include, for example, gut dysbiosis, hormonal disruptions, immune decline, stress, infections, bone loss, injuries, organ malfunction, memory loss and many others.

In one embodiment, the present invention can be used to enhance the functioning of a body system, tissue or organ, such as metabolic functions, the digestive system, the immune system, the endocrine system, and the cardiovascular system.

In one embodiment, the present invention can be used to treat and/or ameliorate the symptoms of health conditions that are a cause and/or a result of a disrupted or unbalanced gut microbiome, such as, for example, Irritable Bowel Syndrome, Type 1 diabetes, Celiac disease, other autoimmune disorders, colorectal cancer, and neurodevelopmental and neurodegenerative diseases, such as ASD and Alzheimer's disease.

In one embodiment, the present invention can be used to treat and/or ameliorate digestive conditions and/or symptoms such as, for example, nausea, vomiting, diarrhea, constipation, gas, bloating, food sensitivities, heartburn, acid-reflux, GERD, indigestion, and abdominal cramps/pain.

In one embodiment, the present invention can be used to treat and/or ameliorate extra-intestinal symptoms associated with a variety of health conditions, including symptoms such as, for example, headaches, dizziness, fatigue, backaches, insomnia, eating disorders, nutrient deficiencies, depression, anxiety, fertility issues, joint or muscle pain, brain fog, genital yeast infections, bacterial vaginosis, bladder or urinary tract infections, and many others.

Growth of Microbes According to the Present Invention

In one embodiment, the methods of the subject invention require efficacious microbe-based compositions comprising high concentrations of one or more beneficial microbial species. Thus, in certain embodiments, the methods further comprise cultivating the benfifical microorganisms isolated from the subject's GI tract.

In preferred embodiments, the subject invention provides methods for cultivating microorganisms and production of microbial metabolites and/or other by-products of microbial growth using a novel form of solid state, or surface, fermentation. Hybrid systems can also be used. As used herein “fermentation” refers to growth of cells under controlled conditions. The growth could be aerobic or anaerobic.

In one embodiment, the present invention provides materials and methods for the production of biomass (e.g., viable cellular material), extracellular metabolites (e.g. small molecules, polymers and proteins), residual nutrients and/or intracellular components (e.g. enzymes and other proteins).

The microbe growth vessel used according to the present invention can be any enclosed fermenter or cultivation reactor for industrial use. In one embodiment, the vessel may optionally have functional controls/sensors or may be connected to functional controls/sensors to measure important factors in the cultivation process, such as pH, oxygen, pressure, temperature, agitator shaft power, humidity, viscosity and/or microbial density and/or metabolite concentration. Preferably, no such controls are necessary, however.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. The oxygenated air may be ambient air supplemented daily through, e.g., air pumps.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors, trace nutrients and/or biostimulants for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate (e.g., ferrous sulfate heptahydrate), iron chloride, manganese sulfate, manganese sulfate monohydrate, manganese chloride, zinc sulfate, lead chloride, copper sulfate, calcium chloride, calcium carbonate, and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the present invention include, but are not limited to, L-alanine, manganese, L-valine, and L-asparagine or any other known germination enhancer.

In some embodiments, the method for cultivation may optionally comprise adding additional acids and/or antimicrobials in to the substrate before and/or during the cultivation process. Advantageously, however, the subject method reduces or eliminates the need for protection from contamination during cultivation due in part to the slower rate of microbial growth.

The pH of the mixture should be suitable for the microorganism of interest, though advantageously, stabilization of pH using buffers or pH regulators is not necessary when using the subject cultivation methods.

In one embodiment, the method for cultivation of microorganisms is carried out at about 15 to 60° C., preferably, 25 to 40° C., and in specific embodiments, 25 to 35° C., or 32 to 37° C. In one embodiment, the cultivation may be carried out continuously at a constant temperature. In another embodiment, the cultivation may be subject to changing temperatures. Temperature can be kept within the preferred range by pumping ambient air into the reactor and circulating it throughout.

In one embodiment, total sterilization of equipment and substrate used in the subject cultivation methods is not necessary. However, the equipment and substrate can optionally be sterilized. The trays can be sterilized before and/or after being spread with nutrient medium, for example, using an autoclave. Additionally, the steam pan lids and pan bands can be sterilized, for example, by autoclaving, prior to inoculation of the solid substrate.

The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control bacterial growth.

In one embodiment, the present invention further provides methods of producing a microbial metabolite by cultivating a microbe strain under conditions appropriate for growth and metabolite production. Optionally, the method can comprise purifying the metabolite. The present invention provides methods of producing metabolites such as, e.g., biosurfactants, biopolymers, ethanol, lactic acid, beta-glucan, proteins, peptides, metabolic intermediates, polyunsaturated fatty acid, lipids and enzymes.

The microbial growth by-product produced by microorganisms of interest may be retained in the microorganisms or secreted into the substrate. The metabolite content can be, for example, at least 20%, 30%, 40%, 50%, 60%, 70%, 80%, or 90%.

In another embodiment, the method for producing microbial growth by-product may further comprise steps of concentrating and purifying the microbial growth by-product of interest. In a further embodiment, the substrate may contain compounds that stabilize the activity of microbial growth by-product.

The method and equipment for cultivation of microorganisms and production of the microbial by-products can be performed in a batch process or a quasi-continuous process.

In one embodiment, all of the microbial cultivation composition is removed upon the completion of the cultivation (e.g., upon, for example, achieving a desired spore density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a cell-free substrate or contain cells. In this manner, a quasi-continuous system is created.

Matrix Fermentation

In preferred embodiments, the present invention provides methods for cultivating microbe-based products using novel procedures and systems for solid state, or surface, fermentation. Advantageously, the present invention does not require fermentations systems having sophisticated aeration systems, mixers, or probes for measuring and/or stabilizing DO, pH and other fermentation parameters.

In preferred embodiments, the method of cultivating a microorganism and/or producing a microbial growth by-product comprises: spreading a layer of a solid substrate mixed with water and, optionally, nutrients to enhance microbial growth, onto a tray to form a matrix; applying an inoculant of the predominant species onto the surface of the matrix; placing the inoculated tray into a fermentation reactor; passing air through the reactor to stabilize the temperature between 25-40° C.; and allowing the predominant species to propagate throughout the matrix.

In preferred embodiments, the matrix substrate according to the subject methods comprises foodstuffs. The foodstuffs can include, for example, rice, beans or legumes, lentils, quinoa, flaxseed, chia, corn, other grains, pasta, wheat bran, flours or meals (e.g., corn flour, nixtamilized corn flour, partially hydrolyzed corn meal), and/or other similar foodstuffs to provide surface area for the microbial culture to grow and/or feed on.

In one embodiment, wherein the matrix substrate comprises pre-made pasta, the pasta can be made from, for example, corn flour, wheat flour, semolina flour, rice flour, quinoa flour, potato flour, soy flour, chickpea flour and/or combinations thereof. In some embodiments, the pasta is made from an enriched flour.

In some embodiments, the pasta can be in the shape of a long string or ribbon, e.g., spaghetti or fettuccini. In some embodiments, the pasta can be in the shape of, for example, a sheet, a shell, a spiral, a corkscrew, a wheel, a hollow tube, a bow, or any variation thereof. Advantageously, the microbes can grow inside the pasta and/or on outside surfaces of the pasta. This increases the surface area upon which the microorganisms can grow, increases the depth of microbial growth within the substrate, and provides enhanced oxygen penetration within the culture.

Other examples of applicable pasta shapes include, but are not limited to, acini di pepe, anelli, angel hair, bucatini, campanelle, cappalletti, cavatappi, casarecce, cavatelli, conchiglie, ditalini, egg noodles, farfalle, farfalline, fettuccine, fideo, fusilli, gemelli, gigli, lasagna, lasagne, linguine, macaroni, mafalda, manicotti, orecchiette, orzo, pappardelle, pastina, penne, pipe rigate, pipette rigate, radiatori, rigatoni, rocchetti, rotelle, rotini, ruote, spaghetti, tagliatelle, tortiglioni, tripolini, tubini, vermicelli, ziti and any variation thereof.

In one embodiment, wherein the matrix substrate comprises grains of rice, the matrix substrate can be prepared by mixing rice grains with water and, depending upon which microbe is being cultivated, an added nutrient medium.

In some embodiments, the rice can be, for example, long grain, medium grain, short grain, white (polished), brown, black, basmati, jasmine, wild, arborio, matta, rosematta, red cargo, sticky, sushi, Valencia rice, and any variation or combination thereof.

In one embodiment, the method of cultivation comprises preparing the trays, which can be, e.g., metal sheet pans or steam pans fitted for a standard proofing oven. In some embodiments, the “trays” can be any vessel or container capable of holding the substrate and culture, such as, for example, a flask, cup, bucket, plate, pan, tank, barrel, dish or column, made of, for example, plastic, metal or glass.

Preparation can comprise covering the inside surfaces of the trays with, for example, foil. Preparation can also comprise sterilizing the trays by, for example, autoclaving them.

Next, a matrix substrate is prepared by mixing a foodstuff item, water, and optionally, additional salts and/or nutrients to support microbial growth. In a specific embodiment, the nutrient medium can comprise, for example, maltose, yeast extract or another source of protein, and sources of minerals, potassium, sodium, phosphorous and/or magnesium.

The mixture is then spread onto the trays and layered to form a matrix with a thickness of approximately 1 to 12 inches, preferably, 1 to 6 inches. The thickness of the matrix can vary depending on the volume of the tray or other container in which is it being prepared.

In preferred embodiments, the matrix substrate provides ample surface area on which microbes can grow, as well as enhanced access to oxygen supply. Thus, the substrate on which the microbes grow and propagate can also serve as the nutrient medium for the microbes.

In some embodiments, grooves, ridges, channels and/or holes can be formed in the matrix to increase the surface area upon which the microorganisms can grow. This also increases the depth of microbial growth within the substrate and provides enhanced oxygen penetration throughout the culture.

To increase the speed of microbial motility throughout the substrate, the method can further comprise applying a biostimulant, potato extract and/or banana peel extract to the substrate. This allows for increased speed of distribution of the culture throughout the surfaces of the substrate.

In some embodiments, when, for example, the microbes used to inoculate the substrate are in spore form, germination enhancers can be applied to the substrate. Examples of germination enhancers according to the present invention include, but are not limited to, L-alanine, manganese, L-valine, and L-asparagine or any other known germination enhancer.

Sterilization of the trays and matrix can then be performed after the matrix has been spread onto the trays. Sterilization can be performed by autoclave or any other means known in the art. In some embodiments, this process will also effectively cook the substrate.

Lids and silicon pan bands can be provided for sealing the trays, if desired. To create a completely sterile system, the lids and pan bands can also be sterilized.

After preparing the matrix substrate in the trays, the trays can be inoculated with a desired microorganism that is optionally pre-mixed with sterile nutrient medium. Optionally, depending upon the microorganism being cultivated and/or the growth by-product being produced, the trays can then be sealed with the lids and pan bands. In one embodiment the trays are not sealed.

The inoculum preferably comprises propagules of the beneficial microorganism(s) collected from the subject's GI tract. The propagules can be vegetative cells, spores or other forms. In one embodiment, inoculation is performed by applying the inoculum uniformly onto the surface of the substrate layer. The inoculum can be applied via, for example, spraying, sprinkling, pouring, injecting or spreading. In one embodiment, inoculation is carried out using a pipette.

The inoculated trays can then be placed inside a fermentation reactor. In one embodiment, the reactor is a proofing oven, such as a standard oven used in commercial baking for, e.g., proofing dough. In one embodiment, the reactor is in the form of a scaled-up enclosure, such as a trailer or a room, that is equipped with the necessary components to provide, for example, tens or hundreds of trays of culture growing on matrix to be incubated at the same time. In one embodiment, the reactor can optionally be equipped with an automated conveyor system or pulley system for continuous production.

In one embodiment, a plurality of reactors can be used, for example, a plurality of proofing ovens. In one embodiment, the reactors are distributable and portable. In a further embodiment, wherein a plurality of reactors is used, the plurality of reactors can be assembled onto a single platform for ease of transport.

Fermentation parameters can be adjusted based on the desired product to be produced (e.g., the desired microbial biosurfactant) and the microorganism being cultivated.

The temperature within the reactor depends upon the microorganism being cultivated, although in general, it is kept between about 25-40° C. using ambient air pumped through the reactor. The circulating air can also provide continuous oxygenation to the culture. The air circulation can also help keep the DO at desired levels, for example, about 90% of ambient air.

In one embodiment, it is not necessary to monitor or stabilize the pH of the culture. The trays may be sprayed regularly throughout fermentation (e.g., once a day, once every other day, once per week) with a sterile nutrient medium for achieving maximum microbial concentration.

The culture can be incubated for an amount of time that allows for the microorganism to reach a desired concentration, or to reach from 50-100% sporulation, preferably from 1 day to 14 days, more preferably, from 2 days to 10 days.

In some embodiments, the microorganisms will consume either a portion of, or the entirety of, the matrix substrate throughout fermentation.

Once the culture sporulates, the culture and remaining substrate can be harvested from the trays, then blended together to produce a microbial slurry. The concentration of microbes grown according to this method can reach, for example, about 1×10⁶ to 1×10¹³ CFU/g, about 1×10⁷ to 1×10¹², about 1×10⁸ to 1×10¹¹, or about 1×10⁹ to 1×10¹⁹.

In one embodiment, the microbial slurry is milled, micronized and/or dried to produce a dry microbe-based product that contains the microorganism, its growth by-products and matrix substrate. The microbial slurry can be dried using any drying method known in the art. In one embodiment, the dried product has approximately 3% to 6% moisture retention.

In one embodiment, the solution containing the dissolved culture is diluted to a concentration of 1×10⁶ to 1×10⁷ CFU/mL using water to form a liquid microbe-based product, which can be utilized in a wide variety of settings and applications. Optionally, nutrients including, e.g., sources of potassium, phosphorous, magnesium, carbon, proteins, amino acids, and others can be added to the water to enhance microbial growth.

Activation and/or germination of spore-form microbes can be enhanced, either during cultivation or at the time of application of the microbe-based product, by adding L-alanine in low (micromolar) concentrations, manganese or any other known germination enhancer.

In one embodiment, the systems and methods of the present invention can be used to produce a microbial metabolite, wherein instead of drying the microbial slurry, the microbial slurry is filtered to separate the liquids from the solids. The liquid that is extracted, which comprises the microbial metabolite, can then be purified further, if desired, using, for example, centrifugation, rotary evaporation, microfiltration, ultrafiltration and/or chromatography.

The metabolite and/or growth by-product can be, for example, a biosurfactant, enzyme, biopolymer, acid, solvent, amino acid, nucleic acid, peptide, protein, lipid and/or carbohydrate. Specifically, in one embodiment, the method can be used to produce a biosurfactant.

Advantageously, the method does not require complicated equipment or high energy consumption. The microorganisms of interest can be cultivated at small or large scale on site and utilized, even being still-mixed with their media. Similarly, the microbial metabolites can also be produced at large quantities at the site of need.

Advantageously, the microbe-based products can be produced in remote locations. The microbe growth facilities may operate off the grid by utilizing, for example, solar, wind and/or hydroelectric power.

Fermentation Room System

In one embodiment, the fermentation reactor utilized in the subject methods can comprise a large, moisture-sealed, enclosed space, having four vertical walls, a floor and a ceiling. The walls can optionally comprise one or more windows and/or doors. This “fermentation room” can replicate the environment that would exist in, for example, a proofing oven fermentation reactor, yet on a much larger scale.

In one embodiment, the fermentation room is fixed onto a portable platform, such as a trailer with wheels.

In one embodiment, the interior walls of the fermentation room have a plurality of horizontal surfaces, upon which the containers for holding inoculated substrate can be placed.

In one embodiment, the surfaces are in the form of shelves. The shelves can be fixed onto the walls of the enclosure. Shelving units can be suspended from the ceiling and/or fixed to the floor.

In one embodiment, the fermentation room comprises a plurality of metal sheet pan racks. The sheet pan racks preferably comprise a plurality of slides for holding trays into which the solid substrate and microbe culture are spread. In one embodiment, the racks are portable, meaning fixed with wheels.

In one embodiment, the pan rack can hold from 10 to 50 trays. Preferably, the slides are spaced at least 3 inches apart from one another to allow for optimal air circulation between each tray.

In one embodiment, the ceiling of the room can optionally be accommodated to allow for air flow, for example, with ceiling vents and/or air filters. Furthermore, the ceiling and walls can be fitted with UV lights to aid in sterilization of air and other surfaces within the system. Advantageously, the use of metal trays and metal pan racks enhances reflection of the UV light for increased UV sterilization.

The room can be equipped with temperature controls, though preferably, the circulation of air throughout the room provides the desired fermentation temperature.

The dimensions of the fermentation room can be customized based on various factors, such as, for example, the location of the room and the number of trays to be placed therein. In one embodiment, the height of the ceiling is at least 8 feet, and the area of the floor is at least 80 square feet.

Beneficial Microbial Culture

In certain embodiments, the present invention provides high concentration microbe-based products comprising one or more microorganisms and/or one or more microbial growth by-products for use in restoring a subject's gut microbiome, wherein the “high” concentration is a concentration of about 1×10⁶ to 1×10¹³ CFU/g, about 1×10⁷ to 1×10¹², about 1×10⁸ to 1×10¹¹, or about 1×10⁹ to 1×10¹⁰. In one embodiment, the composition comprises the matrix substrate containing the microorganism and/or the metabolites produced by the microorganism and/or any residual nutrients from fermentation.

The product of fermentation may be used directly without extraction or purification. If desired, extraction and purification can be achieved using standard extraction methods or techniques known to those skilled in the art.

Upon harvesting of the matrix substrate, microbe, and/or by-products, the product can be dissolved in water to form a liquid product.

Alternatively, upon harvesting of the matrix, microbe and/or by-products, the product can be blended, milled and/or micronized and then dried to form a dry product. This dried product can be dissolved in water and diluted as necessary.

The microorganisms in the microbe-based product may be in an active or inactive form. In some embodiments, the microbes are in vegetative, spore, mycelial, hyphae, conidia form and/or mixtures thereof. The microbe-based products may be used without further stabilization, preservation, and storage.

The dried product and/or liquid product can be transferred for immediate use. In other embodiments, the composition can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 0.5 gallon to 1,000 gallons or more. In certain embodiments the containers are 1 gallon, 2 gallons, 5 gallons, 25 gallons, or larger.

Upon harvesting the microbe-based composition from the reactors, further components can be added as the harvested product is processed and/or placed into containers (or otherwise transported for use). The additives can be, for example, buffers, carriers, other microbe-based compositions produced at the same or different facility, viscosity modifiers, preservatives, nutrients for microbe growth, tracking agents, and other ingredients specific for an intended use.

Advantageously, in accordance with the present invention, the microbe-based product may comprise the substrate in which the microbes were grown. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

Organisms that can be cultured according to the present invention can include, for example, yeasts, fungi, bacteria and archaea, that have been sampled and identified from a subject's GI tract and/or appendix.

In preferred embodiments, the microorganisms are bacteria, such as, for example, Bacteroides spp., Clostridium spp., Faecalibacterium spp., Eubacterium spp., Ruminococcus spp., Peptococcus spp., Peptostreptococcus spp., Enterococcus spp., Bifidobacterium spp., Lactobacillus spp., Enterobacter spp., Klebsiella spp., and Escherichia spp.

In some embodiments, the microorganisms are yeasts or fungi.

In one embodiment, the beneficial microbial culture comprises microbial growth by-products. These can be produced by the microorganisms of the culture, and/or they can be added to the culture prior to its reintroduction into the GI tract. Growth by-products can include, for example, biosurfactants, enzymes, biopolymers, solvents, acids, proteins, amino acids, carbohydrates and/or other metabolites that can be useful for gut restoration.

In one embodiment, the growth by-product is a biosurfactant. Biosurfactants are a structurally diverse group of surface-active substances produced by microorganisms. Biosurfactants are biodegradable and can be produced using selected organisms on renewable substrates. Most biosurfactant-producing organisms produce biosurfactants in response to the presence of a hydrocarbon source (e.g., oils, sugar, glycerol, etc.) in the growing media.

Microbial biosurfactants are produced by a variety of microorganisms, such as, for example, Pseudomonas spp. (P. aeruginosa, P. putida, P. florescens, P. fragi, P. syringae); Flavobacterium spp.; Bacillus spp. (B. subtilis, B. pumillus, B. licheniformis, B. amyloliquefaciens, B. cereus); Wickerhamomyces spp. (e.g., W. anomalus), Candida spp. (e.g., C. albicans, C. rugosa, C. tropicalis, C. lipolytica, C. torulopsis); Rhodococcus spp.; Arthrobacter spp.; Campylobacter spp.; Cornybacterium spp.; Pichia spp. (e.g., P. anomala, P. guilliermondii, P. occidentalis); Starmerella spp. (e.g., S. bombicola); and so on.

All biosurfactants are amphiphiles. They consist of two parts: a polar (hydrophilic) moiety and non-polar (hydrophobic) group. The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), organic acids in the case of flavolipids, or, in the case of glycolipids, by a carbohydrate. Due to their amphiphilic structure, biosurfactants increase the surface area of hydrophobic water-insoluble substances, and increase the water bioavailability of such substances.

Biosurfactants accumulate at interfaces, thus reducing interfacial tension and leading to the formation of aggregated micellar structures in solution. The ability of biosurfactants to form pores and destabilize biological membranes permits their use as antibacterial, antifungal, and hemolytic agents. Combined with the characteristics of low toxicity and biodegradability, biosurfactants are advantageous for use in a variety of application, including in wastewater treatment.

Biosurfactants according to the subject methods can be, for example, glycolipids (e.g., sophorolipids, rhamnolipids, mannosylerythritol lipids, cellobiose lipids, and trehalose lipids), lipopeptides (e.g., surfactin, iturin, fengycin, arthrofactin and lichenysin), flavolipids, phospholipids (e.g., cardiolipins), fatty acid esters, and high , molecular weight polymers such as lipoproteins, lipopolysaccharide-protein complexes, and polysaccharide-protein-fatty acid complexes.

The one or more biosurfactants can further include any one or a combination of: a modified form, derivative, fraction, isoform, isomer or subtype of a biosurfactant, including forms that are biologically or synthetically modified. In certain embodiments, the one or more biosurfactants are applied in pure form. Advantageously, the biosurfactants work in synergy with the enzymes, and/or synergize the different enzymes that are produced by the microbial cocktail to enhance the treatment of the wastewater. These biosurfactants are biodegradable, and thus will degrade, in some embodiments, by the time the wastewater is subjected to aerobic and/or tertiary treatment.

Advantageously, the methods of the subject invention increase the efficiency of treating wastewater by increasing the proportion of beneficial microorganisms in the treatment environment.

In one embodiment, the biosurfactants according to the present invention are glycolipids and/or lipopeptides.

In certain other embodiments, the compositions comprise one or more microbial growth by-products, wherein the growth by-product has been extracted from a microbial culture and, optionally, purified. For example, in one embodiment, the matrix substrate of the subject methods can be blended to form a thick slurry, which can be filtered or centrifuged to separate a liquid portion from a solid portion. The liquid portion, comprising microbial growth by-products, can then be used as-is or purified using known methods.

In one embodiment, the composition can be formulated for administering directly into the GI tract. For example, the composition can be formulated for administration to the proximal lower GI via colonoscopy, the distal lower GI via enema or rectal tubes, and the upper GI tract via nasogastric tubes, duodenal tubes, and endoscopy/gastroscopy.

In one embodiment, the composition can be formulated as an orally-consumable product and administered orally to an animal or human subject.

Orally-consumable products according to the invention are any preparations or compositions suitable for consumption, for nutrition, for oral hygiene or for pleasure, and are products intended to be introduced into the human or animal oral cavity, to remain there for a certain period of time and then to either be swallowed (e.g., food ready for consumption) or to be removed from the oral cavity again (e.g. chewing gums or products of oral hygiene or medical mouth washes). These products include all substances or products intended to be ingested by humans or animals in a processed, semi-processed or unprocessed state. This also includes substances that are added to orally consumable products (particularly food and pharmaceutical products) during their production, treatment or processing and intended to be introduced into the human or animal oral cavity.

Orally-consumable products can also include substances intended to be swallowed by humans or animals and then digested in an unmodified, prepared or processed state; the orally consumable products according to the invention therefore also include casings, coatings or other encapsulations that are intended also to be swallowed together with the product or for which swallowing is to be anticipated.

Local Production of Microbe-Based Products

In certain embodiments of the present invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the present invention can be located at the location where the microbe-based product will be used. For example, the microbe growth facility may be less than 300, 250, 200, 150, 100, 75, 50, 25, 15, 10, 5, 3, or 1 mile from the location of use.

The microbe growth facilities of the present invention produce fresh microbe-based compositions comprising the microbes themselves, microbial metabolites, and/or other components of the medium in which the microbes are grown. If desired, the compositions can have a high density of vegetative cells or propagules, or a mixture of vegetative cells and propagules.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. The system is efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the present invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with a subject's health conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific subject and the subject's health conditions present at the time of application. This provides advantages over compositions that are pre-made in a central location and have, for example, set ratios and formulations that may not be optimal for a given subject.

The microbe growth facilities provide manufacturing versatility by their ability to tailor the microbe-based products to improve synergies with unique subjects. Advantageously, in preferred embodiments, the systems of the present invention harness the power of naturally-occurring gut microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1

### Fermentation of Bacillus Spores

For Bacillus spp. spore production, a wheat bran-based media is used. The media is sterilized in stainless steel steam pans, then sealed with a lid and pan bands. Following sterilization, the pans are inoculated with seed culture and incubated in a proofing oven for 48-72 hours at 32-40° C. At the end of fermentation, 1×10¹⁰ spores/g of Bacillus are harvested.

### Example 2

### Solid State Fermentation of Bacillus subtilis and Bacillus licheniformis

Bacillus subtilis and Bacillus licheniformis can be cultivated using solid state fermentation methods. The medium comprises only corn flour (partially hydrolyzed corn meal) or wheat bran. Optionally, added nutrients can be included to enhance microbial growth, such as, for example, salts, molasses, starches, glucose, sucrose, etc.

Foil-covered trays are autoclaved prior to inoculation. The culture medium is spread on the trays in a layer about 1 to 2 inches thick. Grooves and/or holes are made in the substrate to increase the surface area of the medium. To increase the speed of growth, i.e., increase the motility of the bacteria and distribution throughout the culture medium, potato extract or banana peel extract can be added to the culture.

Spores of the Bacillus strain of choice are then sprayed onto the surface of the substrate and the trays are placed into a proofing oven. Fermentation inside the proofing oven occurs at a temperature between 32-40° C. Ambient air is pumped through the oven to stabilize the temperature.

The concentration of microbes grown according to this method when dissolved in water can reach at least 5×10⁹ to 5×10¹⁰ spores/ml. The product is then diluted with water in a mixing tank to a concentration of 1×10⁶ to 1×10⁷ spores/ml. Nutrients that can also be added include, e.g., potassium salts (0.1% or lower), molasses and/or glucose (1-5 g/L), and nitrates.

### Example 3

### Fermentation of Bacillus subtilis for Iturin A Production

A nutrient medium comprising the following components is prepared for growing Bacillus subtilis for iturin A production:
Mixture of polished rice and water (1:1.25, rice to water)
Soybean meal and/or corn step solids (80 g/L)
Maltose (67 g/L)
Potato extract (1%).

The nutrient medium components are mixed and placed in a container fitted with an air filter for aeration, then inoculated with B. subtilis. The containers, rice, water, and optional nutrients can then be sterilized by, for example, autoclaving. This process effectively cooks the rice and creates a porous, sticky substrate. After preparation and sterilization, the containers are inoculated with a desired microorganism.

Fermentation is carried out in an incubator at 37° C. for 4 to 14 days. The fermentation medium and microorganisms are blended into a thick slurry and pressed through a filter to produce a liquid supernatant comprising microbial growth by-products, e.g., iturin A. This liquid can be centrifuged, or purified by other known means to extract and purify the iturin A.

### REFERENCES

Clemente, J. C., Ursell, L. K., Parfrey, L. W., & Knight, R. (2012). The Impact of the Gut Microbiota on Human Health: An Integrative View. Cell, 148(6), 1258-1270. doi:10.1016/j.cell.2012.01.035. (“Clemente 2012”).

Turnbaugh, P. J., Quince, C., Faith, J. J., McHardy, A. C., Yatsunenko, T., Niazi, F., Affourtit, J., Egholm, M., Henrissat, B., Knight, R., and Gordon, J. I. (2010). Organismal, genetic, and transcriptional variation in the deeply sequenced gut microbiomes of identical twins. Proc. Natl. Acad. Sci. USA 107, 7503-7508. (“Turnbaugh 2010”).

Tannock, G. W. (2003). The Intestinal Microflora. In R. Fuller & G. Perdigon (Eds.), Gut Flora, Nutrition, Immunity and Health (pp. 1-23). Malden, USA: Blackwell Publishing Ltd. (“Tannock 2003”).

Lozupone, C., Stombaugh, J., Gordon, J., Jansson, J., & Knight, R. (2012). Diversity, stability and resilience of the human gut microbiota. Nature, 489(7415), 220-230. doi: 10.1038/nature11550. (“Lozupone 2012”).

Benson, A. K., Kelly, S. A., Legge, R., Ma, F., Low, S. J., Kim, J., Zhang, M., Oh, P. L., Nehrenberg, D., Hua, K., et al. (2010). Individuality in gut microbiota composition is a complex polygenic trait shaped by multiple environmental and host genetic factors. Proc. Natl. Acad. Sci. USA 107, 18933-18938. (“Benson 2010”).

Nagpal R, Mainali R, Ahmadi S, et al. Gut microbiome and aging: Physiological and mechanistic insights. Nutrition and Healthy Aging. 2018; 4(4):267-285. doi:10.3233/NHA-170030. (“Nagpal 2018”).

1. A method for restoring a subject's gut microbiome, the method comprising:
taking a sample from the subject's gastrointestinal (GI) tract and/or appendix, wherein the sample comprises a microbial community;
analyzing the sample to determine the identity of microbial species present and the population percentage of each species within the community;
determining which microbial species within the community are undesirable and which microbial species are beneficial, and isolating the beneficial species;
cultivating the beneficial species to produce a microbial culture with increased cell concentration; and
reintroducing the microbial culture with increased cell concentration into the subject's GI tract,
wherein the ratio of beneficial microbial species to undesirable microbial species in the subject's GI tract increases.

2. The method of claim 1, wherein the sample is a stool sample, an intestinal mucosal lavage sample, an appendix tissue specimen or an intestinal tissue specimen.

3. The method of claim 1, further comprising cleansing the subject's GI tract using colon hydrotherapy prior to reintroducing the beneficial species.

4. The method of claim 1, further comprising administering prebiotics to the subject before, concurrently with, and/or after reintroducing the beneficial species.

5. The method of claim 1, wherein the subject is a human whose gut microbiome has been disrupted or unbalanced.

6. The method of claim 5, wherein the gut microbiome has been disrupted or unbalanced as a result of antibiotic treatment, illness, infection, dietary factors, colonoscopy, appendectomy, and/or aging.

7. The method of claim 1, further comprising introducing a microbial growth by-product into the subject's GI tract with the beneficial species.

8. The method of claim 7, wherein the microbial growth by/product is a biosurfactant.

9. The method of claim 8, wherein the biosurfactant is a glycolipid selected from sophorolipids, rhamnolipids, trehalose lipids, cellobiose lipids and mannosylerythritol lipids.

10. The method of claim 8, wherein the biosurfactant is a lipopeptide selected from a surfactin, iturin, fengycin, arthrofactin and lichenysin.

11. (canceled)

12. The method of claim 1, wherein the beneficial species are reintroduced via oral consumption.

13. The method of claim 1, wherein the subject's health and well-being is enhanced

14. The method of claim 1, wherein the severity of a health condition resulting from aging and/or senescence in the subject is reduced.

15. The method of claim 1, wherein the functioning of the subject's digestive system, immune system, endocrine system or cardiovascular system is enhanced.

16. The method of claim 1, wherein a symptom of Irritable Bowel Syndrome, Type 1 diabetes, Celiac disease, colorectal cancer, a neurodevelopmental disease or neurodegenerative disease in the subject is ameliorated.

17. The method of claim 1, wherein a digestive condition and/or digestive symptom selected from nausea, vomiting, diarrhea, constipation, gas, bloating, food sensitivities, heartburn, acid-reflux, GERD, indigestion, and abdominal cramps/pain in the subject is ameliorated.

18. The method of claim 1, wherein an extra-intestinal symptom associated with a health condition in the subject is ameliorated, said symptom selected from headaches, dizziness, fatigue, backaches, insomnia, eating disorders, nutrient deficiencies, depression, anxiety, fertility issues, joint or muscle pain, brain fog, genital yeast infections, bacterial vaginosis, and bladder or urinary tract infections.

19-35. (canceled)

36. The method of claim 1, wherein the beneficial species is a bacterium selected from Bacteroides spp., Clostridium spp., Faecalibacterium spp., Eubacterium spp., Ruminococcus spp., Peptococcus spp., Peptostreptococcus spp., Enterococcus spp., Bifidobacterium spp., Lactobacillus spp., Enterobacter spp., Klebsiella spp., and Escherichia spp.
